(19) United States
(12) Patent Application Publication
(10) Pub. No.: US 2009/0175408 A1
(43) Pub. Date: Jul. 9, 2009

(54) COMPRESSION PADDLE AND METHODS FOR USING THE SAME IN VARIOUS MEDICAL PROCEDURES

(76) Inventors: Mitchell M. Goodsitt, Ann Arbor, MI (US); Daniel J. March, Fraser, MI (US); Jonathon T. Ong, West Bloomfield, MI (US); Jeffrey Grimm, Chicago, IL (US); James Pavlik, Clawson, MI (US); Nancy J. Thorson, South Lyon, MI (US)

Correspondence Address: JULIA CHURCH DIERKER;DIERKER & ASSOCIATES, P.C., 3331 W. BIG BEAVER RD. SUITE 109, TROY, MI, 48084-2813, US

(21) Appl. No.: 12/328,510

(22) Filed: Dec. 4, 2008

Related U.S. Application Data
(60) Provisional application No. 60/992,304, filed on Dec. 4, 2007.

(51) Int. Cl. A61B 6/04; H05G 1/00

(52) U.S. Cl. 378/37; 378/208

(57) ABSTRACT

A compression paddle includes a frame defining a hollow cavity. The cavity has two opposed ends. A plurality of filaments is attached to at least one of the two opposed ends of the cavity such that the plurality of filaments extends across the cavity in a predetermined manner.

### CROSS-REFERENCE TO RELATED APPLICATIONS

This application claims the benefit of U.S. Provisional Patent Application Ser. No. 60/992,304 filed Dec. 4, 2007, which is incorporated herein by reference in its entirety.

### STATEMENT REGARDING FEDERALLY SPONSORED RESEARCH OR DEVELOPMENT

Embodiments of this invention were made in the course of research partially supported by a grant from the National Institutes of Health, Grant Number RO1 CA91713-01. The U.S. government has certain rights in the invention.

### BACKGROUND

The present disclosure relates generally to compression paddles.

Compression paddles used in mammography and other image obtaining procedures are often formed of solid materials. Those employed in specialized procedures, such as wire localization and breast biopsy, have an aperture defined therein. Solid compression paddles with apertures require that the object (e.g., breast) be positioned accurately relative to the aperture, such that the desirable area is exposed through the aperture. Design restrictions of solid compression paddles with or without apertures may, in some instances, contribute to accessibility limitations and/or image quality limitations.

### SUMMARY

A compression paddle is disclosed herein. A compression paddle includes a frame defining a hollow cavity. The cavity has two opposed ends. A plurality of filaments is attached to at least one of the two opposed ends of the cavity such that the plurality of filaments extends across the cavity in a predetermined manner.

### BRIEF DESCRIPTION OF THE DRAWINGS

Features and advantages of embodiments of the present disclosure will become apparent by reference to the following detailed description and drawings, in which like reference numerals correspond to the same or similar, though perhaps not identical, components. For the sake of brevity, reference numerals having a previously described function may or may not be described in connection with subsequent drawings in which they appear.

FIG. 1 is a semi-schematic top perspective view of an embodiment of a compression paddle having crisscrossed filaments;

FIG. 2 is a semi-schematic top perspective view of an embodiment of a compression paddle having filaments positioned perpendicular to an object (e.g., a chest wall);

FIG. 3 is a semi-schematic top perspective view of an embodiment of a compression paddle having a wire localization grid removeably placed thereon;

FIG. 4 is a semi-schematic top perspective view of an embodiment of a compression paddle having filaments positioned parallel to an object (e.g., a chest wall);

FIG. 5 is a semi-schematic top perspective view of an embodiment of a compression paddle having filaments positioned at an angle;

FIG. 6 is a semi-schematic top perspective view of an embodiment of a compression paddle having some filaments positioned in a first direction and other filaments positioned in a second direction;

FIG. 7 is a semi-schematic top perspective view of another embodiment of a compression paddle having some filaments positioned in a first direction and other filaments positioned in a second direction;

FIG. 8 is a semi-schematic top perspective view of an embodiment of a compression paddle having two sets of filaments positioned a spaced distance apart;

FIG. 9 is a semi-schematic side perspective view of an embodiment of a compression paddle having sides with varying depths;

FIG. 10 is a semi-schematic top perspective view of an embodiment of a compression paddle having filaments suitable for combined ultrasound imaging and x-ray imaging; and

FIG. 11 is a schematic side view of an embodiment of a rigid compression paddle attached to a device to obtain a desirable tilt angle.

### DETAILED DESCRIPTION

Embodiments of the compression paddle disclosed herein are believed to increase the area available for wire localization, to minimize vertical skin deflection, and to increase the ease of use. The compression paddle(s) may advantageously be used to obtain medical images using ultrasound imaging, magnetic resonance imaging, optical imaging, thermography imaging, scintigraphy imaging, microwave imaging, photoacoustic imaging, thermoacoustic imaging, x-ray imaging, digital mammography imaging, tomosynthesis imaging, CT imaging, breast CT imaging, elasticity imaging, electrical impedance imaging, PET imaging, and combinations thereof. Examples of medical procedures in which the compression paddle may be used include, but are not limited to, wire localizations, breast biopsies, hyperthermia treatments (e.g., thermoablation), and cryogenic treatments (e.g., cryoablation). It is to be understood that any of the above techniques may be used in combination, for example, the compression paddle may be useful for combined ultrasound and x-ray imaging.

Any of the previously listed techniques may be performed through the paddle disclosed herein (i.e., in most instances, the paddle need not be removed or moved in order to perform the desired procedure). As such, the compression paddle disclosed herein may have applied directly thereon any gels or other substances suitable or necessary for performing the particular procedure. Without being bound to any theory, it is believed that the filaments used in embodiments of the compression paddle will also achieve sufficient compression and minimize or eliminate artifacts and other deleterious effects in the obtained images. Still further, the filaments of the compression paddle(s) disclosed herein increase the available area of the surface to be imaged and do not require special positioning on the surface to be imaged (e.g., a human breast).

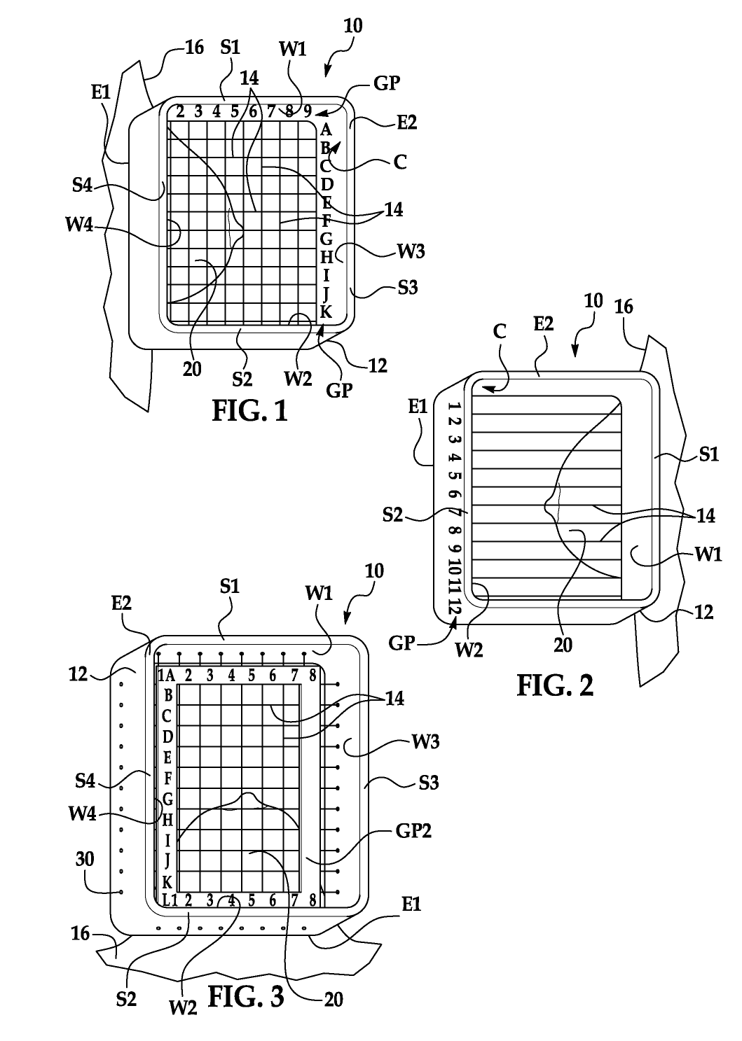

Referring now to FIG. 1, an embodiment of the compression paddle 10 is depicted. Generally, the compression paddle 10 includes a frame 12 defining a hollow cavity C. The frame 12 may have any suitable size, shape and/or configuration. In an embodiment, the frame 12 is configured such that the cavity C has a substantially rectangular cross-section. In another embodiment, the frame 12 is configured such that the cavity C has a substantially square cross-section. It is to be understood that the size, shape and/or configuration of the frame 12 (and thus the cavity C) may be altered depending, at least in part, on the object or surface 20 to be imaged. In some instances, the cavity C has a substantially circular or oval cross-section.

The cavity C has two opposed ends E1, E2. A plurality of filaments 14 are attached to at least one of the two opposed ends E1, E2 of the cavity C such that the filaments 14 extend across the cavity C in a predetermined manner. In some instances (not shown in the Figures), it may be desirable to have some filaments 14 attached to one of the ends E1, E2, and to have other filaments 14 attached to the other of the ends E2, E1.

In the embodiment shown in FIG. 1, the predetermined manner is a crisscross pattern. For example, some of the filaments 14 cross at least some other of the filaments 14 to form the crisscross pattern across the cavity C. It is to be understood that the crossing filaments 14 may be intertwined (e.g., similar to tennis racket strings).

As shown in FIG. 1, the frame 12 has at least two opposed sides S1, S2, S3, S4. While each of the Figures disclosed herein illustrates four sides S1, S2, S3, S4 (two of which are positioned opposite each other) forming a substantially rectangular shape, it is to be understood that any number of sides S1, S2, S3, S4 may define the cavity C of the frame 12. Each side S1, S2, S3, S4 defines a respective wall W1, W2, W3, W4 of the cavity C. As such, this embodiment of the compression paddle 10 includes two sets of opposed walls W1, W2, W3, W4 as well. The embodiment of FIG. 1 includes some of the filaments 14 extending in a first direction from one wall W1 (side S1) to an opposed wall W2 (side S2), and some other of the filaments 14 extending in a second direction substantially perpendicular to the first direction and from another wall W3 (side S3) to an opposed wall W4 (side S4). As previously stated, this forms crisscross pattern of the filaments 14. It is to be understood that other filament 14 configurations (i.e., predetermined manners) are shown and discussed in reference to the other Figures.

In the embodiments disclosed herein, the filaments 14 are securely attached to the end E1, E2 and/or walls/sides W1, S1, W2, S2, W3, S3, W4, S4 (or other areas of the frame 12, described further hereinbelow) such that deflection of a respective filament 14 is minimized upon exposure to an object or surface 20. As non-limiting examples, the filaments 14 may be attached to the frame 12 via stringing the filaments 14 through small holes (not shown) in the frame 12 or winding the filaments 14 around small pegs (also not shown) attached to or incorporated in the frame 12. As a non-limiting example, the filaments 14 may be attached to the frame 12 similarly to how strings are attached to a tennis racket. For a one-piece mesh design (discussed below), means are provided for attaching the mesh to each side S1, S2, S3, S4 of the frame 12 and for maintaining the mesh in a stretched condition (e.g., via a bar or clamp system). When a one-piece mesh design is utilized, it is to be understood that the frame 12 may include multiple pieces between which the one-piece mesh is secured.

In one example, the frame 12 may include two or more cylindrical pieces which define slots therebetween. The edges of the one-piece mesh may be inserted in the slots and held firmly therein with a spline. The cylinders may then be rotated to tighten the mesh in the frame 12. This is similar to a frame used in silk screening. A non-limiting example of a suitable frame for use in silk screening is a Newman Roller M-1 Cap Frame (8.5″×12″).

It is to be understood that another method of attaching the one-piece mesh to the frame 12 is accomplished without rotating cylinders. This may be accomplished by inserting and affixing the mesh in one or more grooves within the frame 12 with one or more splines. This method (without rotating cylinders) is similar to a method employed for attaching window screens to their frames.

In still another example, the frame 12 may include two interlocking pieces (e.g., similar to an embroidery hoop). The one-piece mesh may be established over the edges of the smaller interlocking piece, and the larger interlocking piece may be established over the one-piece mesh such that the smaller interlocking piece (having the mesh thereon) is surrounded by the larger interlocking piece. The larger interlocking piece may be tightened around the mesh and the smaller interlocking piece via, for example, a screw fitting operatively disposed on the larger interlocking piece. The edges of the one-piece mesh may then be pulled in order to stretch it to a desirable configuration (similar to tightening a membrane on a drumhead).

Generally, in the embodiments disclosed herein, the filaments 14 have a size and shape that minimize the presence of air bubbles and gaps between the filaments 14 and the object or surface 20 being compressed via the paddle 10.

It is to be understood that the filaments 14 may be formed of any suitable material. In an embodiment, the filaments 14 are incorporated into a one-piece mesh material. For the one-piece mesh material embodiments disclosed herein, it may be desirable that the mesh be weaved such that the filaments are denser (i.e., more filaments per unit area) at the edges of the mesh material where it will be secured to the frame 12. Still further, when using the one-piece mesh material, it may be desirable to use a different material (e.g., cloth) at the edges where the mesh material will be secured to the frame 12. Such a different material may be integrally formed with the mesh material or may be otherwise secured (e.g., via glue) to the mesh material. It is believed that, in some instances, such a different material will provide better contact for the mesh material with the frame 12. In another embodiment, a mesh material (incorporating the filaments 14) and the frame 12 are all one piece. In still another embodiment, the filaments 14 are formed of a substantially flat material, similar to dental floss or dental ribbon. It may be desirable to utilize filaments 14 that are one or more of hypoallergenic, water resistant, coupling agent resistant, and capable of being sterilized.

The filament 14 material is generally selected to exhibit characteristics that result in minimal undesirable or deleterious effects and artifacts in medical images obtained through the paddle 10. The filament 14 material may also advantageously be invisible in the obtained image. In some instances, the filament 14 material may not have ideal acoustic properties, but has a very small diameter such that it has minimal effects on, for example, ultrasound waves. In other instances, the filament 14 material exhibits desirable acoustic characteristics for imaging through the paddle 10. More specifically, the filament 14 material may exhibit characteristics selected from minimal absorption of ultrasound waves, an acoustic impedance that minimizes ultrasound reflection, a speed of sound that minimizes refraction, a coefficient of friction that minimizes slippage, a predetermined tensile strength, and combinations thereof.

The size (e.g., diameter) and configuration (i.e., spacing, pattern formed, etc.) of the respective filaments 14 may depend, at least in part, on the ultimate end use of the paddle 10. As a non-limiting example, thinner filaments 14 may be more desirable when the paddle 10 is used for wire localization. Non-limiting examples of filament 14 diameter range from about 0.1 mm to about 0.5 mm.

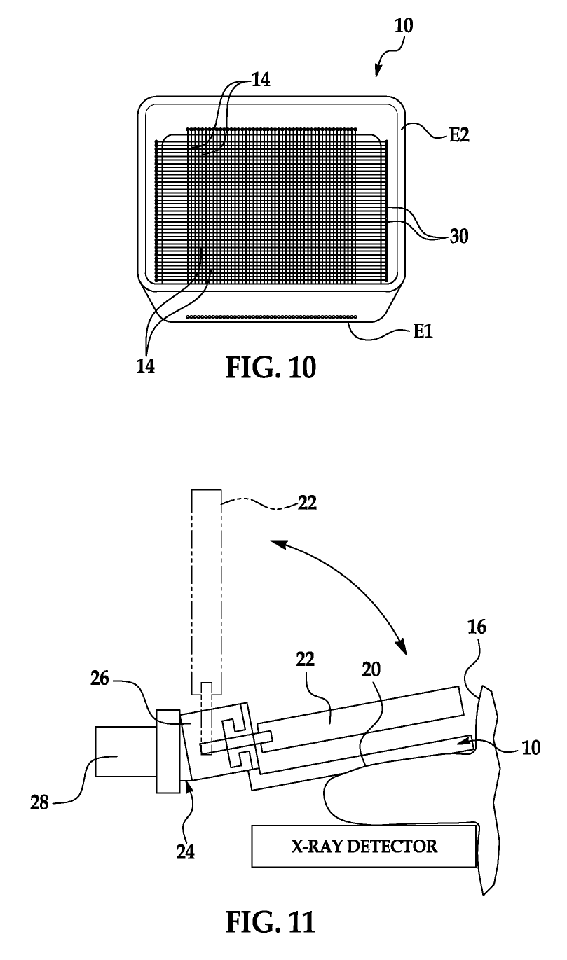

The spacing of each filament 14 with respect to each adjacent filament 14 may depend, at least in part, on the ultimate end use of the paddle 10. In some instances, such as, for example, in combined ultrasound and x-ray imaging, it may be desirable to have adjacent filaments 14 relatively close together (e.g., less than 5 mm apart). A non-limiting example of such close spacing is semi-schematically shown in FIG. 10. In other instances, such as, for example, in wire localization, it may be desirable to have adjacent filaments 14 relatively far apart (e.g., equal to or greater than 1 cm apart). As a non-limiting example, the spacing between adjacent filaments 14 ranges from about 1 mm to about 1 cm. Specific non-limiting examples of suitable spacing for adjacent filaments 14 include 1.2 mm, 3 mm, 6 mm, and 9 mm. It is to be understood that the spacing between adjacent filaments 14 may be consistent across the cavity C (e.g., each filament 14 is 3 mm from each adjacent filament 14), or may vary across the cavity C (e.g., some adjacent filaments 14 are 1.5 mm apart and other adjacent filaments 14 are 3 mm apart). Furthermore, the filament 14 spacings disclosed herein are examples, and other desirable spacing configurations may be employed in the embodiments disclosed herein.

Without being bound to any theory, it is believed that the various configurations (some of which are discussed further hereinbelow) of the compression paddle 10 increase the area of the object or surface 20 available for treatment (e.g., when compared to a solid compression paddle with an aperture). This is due, at least in part, to the fact that the areas between the respective filaments 14 area available for object or surface 20 exposure. As a non-limiting example, a radiologist may insert a needle at any desired location between the filaments 14. This essentially increases the area available for performing a wire localization procedure.

Referring now to FIG. 2, another embodiment of the compression paddle 10 is depicted. The object or surface 20 shown being compressed by the paddle 10 is a human breast. As depicted, this embodiment of the frame 12 includes two opposed sides S1, S2 that are configured to align substantially parallel with a particular object 16. In this embodiment, the object 16 is the chest wall of the person being treated with the paddle 10. It is to be understood that the object 16 and sides S1, S2 may not be exactly parallel, but are generally positioned such that they extend in the same or similar direction.

In this embodiment, the filaments 14 are arranged substantially parallel with respect to each other. Furthermore, the predetermined manner in which the filaments 14 extend across the cavity C is perpendicular to the sides S1, S2. As such, when the paddle 10 is in use, the filaments 14 are aligned substantially perpendicular with the object 16. It is to be understood that object 16 and filaments 14 may not be exactly perpendicular, but are generally positioned such that they extend in different directions that are close to 90° apart.

FIGS. 1 and 2 illustrate two different embodiments of a grid plate GP which may be operatively disposed on (or otherwise connected to) the frame 12. Generally, the grid plate GP is positioned such that it may be seen in the resulting images and/or by a technician performing the medical procedure. Such positioning enables one reading the images and/or performing the medical procedure to determine the coordinates for insertion of, for example, a wire of needle. The grid plate GP may be etched in, printed on, or other established in/on the frame. The grid plate GP may include numbers, letters, graphics, or any other desirable indicia, or any combination thereof. As shown in FIG. 1, the grid plate GP includes letters and numbers on the walls W1, W3, respectively, identifying columns and rows defined between adjacent filaments 14. As shown in FIG. 2, the grid plate GP includes letters on the outside of wall W2 identifying rows defined between adjacent filaments 14. It is to be understood that the grid plate GP may also be positioned on one of the ends E1, E2, as long as the grid plate GP is visible during paddle 10 use.

Referring now to FIG. 3, another embodiment of the compression paddle 10 is depicted. In this embodiment, the compression paddle 10 is associated with an add-on grid GP2. In some instances, the add-on grid GP2 is permanently attached to the frame 12; in other instances, the add-on grid GP2 is an integral part of the frame 12; and in still other instances, the add-on grid GP2 is readily attachable and removable from the frame 12. The add-on grid GP2 may include leaded letter and numbers that are positioned in a pattern similar to that shown in FIG. 3. It is to be understood that the leaded numbers and letters may also be placed on, adhered to, or etched into a material, such as aluminum or polymeric materials. It is to be still further understood that the letters and numbers may be made of other materials that are highly attenuating for x-rays, such as copper. Any etching of the letters, numbers, or other identifying indicia in the grid GP2 should be deep enough to obtain sufficient contrast in the image.

The letters of the grid GP2 in FIG. 3 are positioned adjacent to the walls W1, W2 that are respectively opposite from and directly adjacent to the chest wall (object 16), and the numbers are positioned adjacent to the other two walls W3, W4. It is to be understood that the letters and numbers may be configured on the add-on grid GP2 in any desirable manner.

When using a readily attachable and removable add-on grid GP2, as shown in FIG. 3, the add-on grid GP2 is placed above the filaments 14 and inside the 12 frame of the compression paddle 10. In one non-limiting example, the add-on grid GP2 is formed of 1/16 of an inch thick aluminum, which may be desirable for compressed breasts that are 2-4 cm thick. In another non-limiting example, the add-on grid GP2 in the previous example includes a second 1/16 of an inch think layer of aluminum, which may be desirable for thicker compressed breasts. In another embodiment, two add-on grids GP2 may be used together, one of which is 1/16 of an inch thick and the other of which is ⅛ of an inch thick. The width of the grid strips should be about 1 cm or less to minimize interference with the image of the object 16, thereby leaving a maximum open area for imaging or performing another procedure on the object 16.

FIG. 3 also illustrates small holes 30 in the frame 12, through which the filaments 14 are attached to the frame 12.

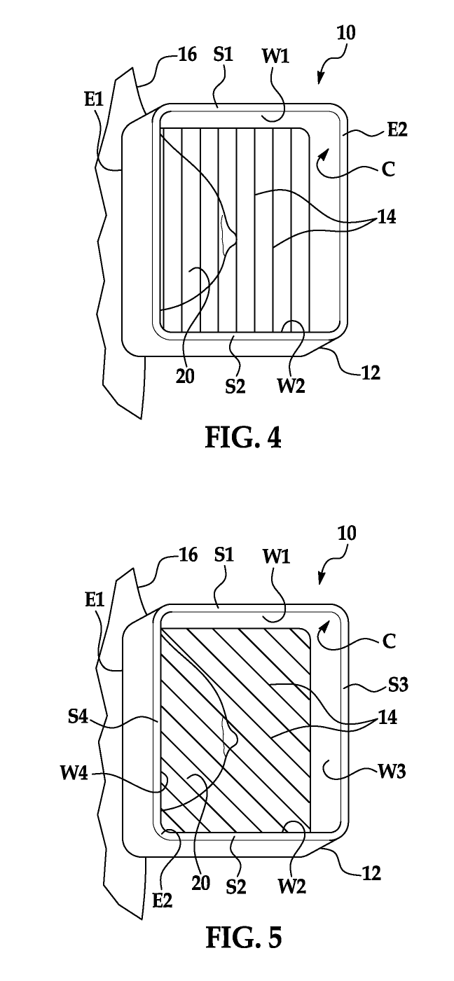

Referring now to FIG. 4, still another embodiment of the compression paddle 10 is depicted. Again, the object or surface 20 shown being compressed by the paddle 10 is a human breast. As depicted, this embodiment of the frame 12 includes two opposed sides S1, S2 that are configured to align substantially perpendicular with a particular object 16. In this embodiment, the object 16 is again the chest wall of the person being treated with the paddle 10. It is to be understood that the object 16 and sides S1, S2 may not be exactly perpendicular, but are generally positioned such that they extend in different or opposite directions that are close to 90° apart.

In this embodiment, the filaments 14 are arranged substantially parallel with respect to each other. Furthermore, the predetermined manner in which the filaments 14 extend across the cavity C is perpendicular to the sides S1, S2. As such, when the paddle 10 is in use, the filaments 14 are aligned substantially parallel with object 16. It is to be understood that object 16 and filaments 14 may not be exactly parallel, but are generally positioned such that they extend in the same or similar direction.

FIG. 5 depicts still another embodiment of the compression paddle 10. In this embodiment, the cavity C has a substantially rectangular shape and is defined by two sets of opposed walls W1, W2, W3, W4. As depicted, walls W1, W2 are opposed to each other, and walls W3, W4 are opposed to each other. The predetermined manner in which the filaments 14 are arranged includes each of the plurality of filaments 14 being angularly offset with respect to each of the walls W1, W2, W3, W4 of the rectangular cavity C. It is to be understood that the filaments 14 may be positioned at any desirable angle with respect to the walls W1, W2, W3, W4. Non-limiting examples of such angles includes 30°, 45°, 60°, 120°, 135°, 150° or the like. While FIG. 5 depicts all of the filaments 14 positioned at about the same angle, it is to be understood that different filaments 14 may be positioned at two or more different angles. For example, some filaments 14 may be attached at 45°, while other filaments 14 may be attached at 60°. While not shown in FIG. 5, the filaments 14 positioned at two or more different angles may also cross one another.

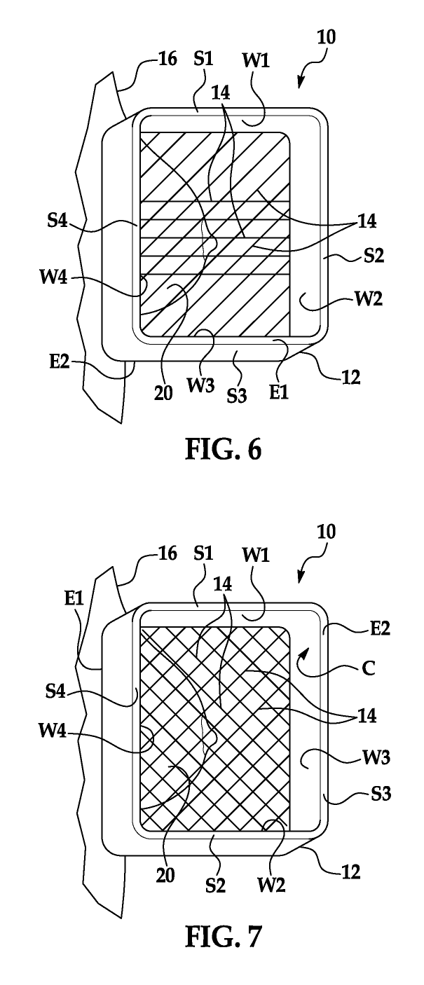

Referring now to FIGS. 6 and 7, two different embodiments of the compression paddle 10 having filaments 14 extending in different directions are respectively depicted. In FIG. 6, some of the filaments 14 are angularly offset from one or more of the walls (e.g., W4) in one direction (e.g., 0°, 30°, 45°, 60°, 120°, 135°, or 150°), and other filaments 14 are positioned parallel with sides S1, S3 and perpendicular with sides S2, S4. It is to be understood that the other filaments 14 could be angularly offset in another direction, as is desired. For example, in FIG. 7, some of the filaments 14 are angularly offset at 45° with respect to walls W2, W3, and other filaments 14 are angularly offset at −45° (135°) with respect to walls W2, W3. This embodiment forms crossing angled filaments 14 that are approximately 90° apart. It is to be understood that in this embodiment, the crossing angled filaments 14 are not limited to being 90° apart and may be any desirable angle apart. The compression paddle 10 shown in FIG. 7 may be particularly suitable for use with a combined x-ray and ultrasound imaging system. The embodiments shown in FIGS. 6 and 7 are illustrative, and it is to be understood that the different filaments 14 may extend and/or cross in any number of different directions with respect to each other.

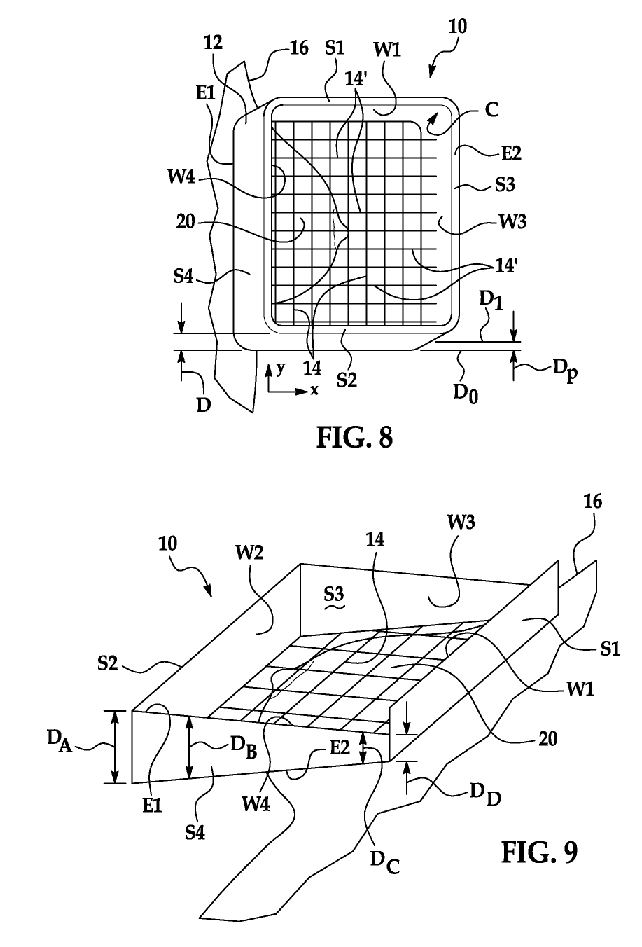

FIG. 8 depicts still another embodiment of the compression paddle 10. In this embodiment, the first set of filaments 14 is attached to the end E1 and extends across the cavity C from side S1/wall W1 to side S2/wall W2. While the first set of filaments 14 is shown attached to end E1, it may, in another embodiment, be attached to end E2.

The compression paddle shown in FIG. 8 includes a second set of filaments 14′ attached to at least two walls W3, W4 of the cavity C. The second set of filaments 14′ extends across the cavity C in a second predetermined manner, which may be the same of different than the plurality of filaments 14 attached to the end E1. In the embodiment of FIG. 8, the filaments 14, 14′ are perpendicular to each other. The X, Y graph is shown in the Figure simply to illustrate the positioning of the filaments 14, 14′ with respect to the object 16 and with respect to each other (i.e., some filaments 14 are parallel with the object 16 (both extending along the Y-axis) and other filaments 14′ (extending along the X-axis) are perpendicular to the object 16 (extending along the Y-axis)).

It is to be understood that since the filaments 14′ of the second set are attached to the walls W3, W4 of the cavity C, each filament 14′ is positioned a predetermined distance DP from the end E1 of the cavity C to which the first set of filaments 14 is attached.

In the embodiment shown in FIG. 8, the cavity C has a depth D, and the end E1 to which filaments 14 are attached is located at depth D₀. The filaments 14′ attached to the walls W3, W4 are located at depth D₁, which is a predetermined distance Dₚ from depth D₀. It is to be understood that the second set of filaments 14′ may be positioned on any of the walls W1, W2, W3, W4 and at any predetermined distance D_{P} from depth D₀. In some embodiments, the predetermined distance D_{P} ranges from about 0.5 mm to about 2 mm.

Generally, the depth D does not have to be very deep, and the thickness of the frame 12 may be fairly thin (e.g., 5 mm). As a non-limiting example, the depth D may range from about 1.5 cm to about 4 cm. Furthermore, the depth D may vary for each of the respective sides S1, S2, S3, S4. A non-limiting example of this is shown in FIG. 9. As depicted, the paddle 10 of FIG. 9 has a fixed tilt due to the varying depth D_{A}, D_{B}, D_{C}, D_{D} of angled sides S3, S4 of the frame 12. Generally, the depth of the sides S3, S4 decreases between the other two sides S1, S2 (e.g., as shown in FIG. 8, the depths D_{A}, D_{B}, D_{C}, D_{D} decrease as one moves along the side S3, S4 from side S2 to side S1).

As shown in FIG. 9, this embodiment of the compression paddle 10 also includes side S1 extending beyond the end E2 of the frame 12. It is to be understood that any side S1, S2, S3, S4 positioned nearest the object 16 may include this extension. Without being bound to any theory, it is believed that this extension keeps loose skin from folding over the paddle 10 into the imaging area. Furthermore, it is believed that the thinness of the frame 12 enables imaging of the surface 20 close to the object 16 (e.g., breast tissue close to chest wall).

It is to be understood that for the embodiments disclosed herein, the filaments 14, 14′ are generally attached to the end E1, E2 that contacts the surface 20 to be imaged. This ensures that the filaments 14, 14′ come in contact with the surface 20 at about the same time as the end E1, E2 of the frame 12 comes in contact with the surface 20.

Without being bound to any theory, it is believed that the embodiment shown in FIG. 8 may be particularly suitable for sterilizing the filaments 14, 14′, as they are spaced apart and may be fully exposed to a cleaning solution. While the embodiment shown in FIG. 8 may be advantageous for cleaning purposes, it is to be understood that any of the embodiments of the compression paddle 10 disclosed herein may be sterilized. Generally, a cleaning solution that does not deleteriously affect the filaments 14, 14′ may be used.

In any of the embodiments disclosed herein, it is to be understood that the frame 12 may be formed of a relatively rigid material, or a relatively flexible material. It may be desirable to use a flexible material such that the frame 12 flexes or tilts in one or more directions (e.g., from the chest wall 16 to the anterior portion of the breast) when in contact with the surface 20.

FIG. 10 depicts still another embodiment of the compression paddle 10. In this embodiment, the filaments 14 are crossing angled filaments 14 that are approximately 90° apart (although could be at any other desirable angles). The spacing between the filaments 14 in this embodiment is very small, and thus is particularly suitable for combined x-ray and ultrasound imaging techniques, and may not be suitable for wire localization techniques.

FIG. 11 depicts an embodiment of a rigid compression paddle 10 attached, via a block 26, to a mechanism 28 for moving the paddle 10 up and down. Positioned between the block 26 and the mechanism 28 is a wedge 24 (or other like mechanism), which enables the paddle 10 to be tilted at a fixed desirable angle. The size of the wedge 24 may be varied to obtain a desirable angle. Still further, the paddle 10 may be configured such that two of the sides S1, S2, S3, S4 are positioned at an angle relative to the x-ray detector plane (e.g., the plane containing the filaments 14 is at an angle relative to the plane of the x-ray detector).

A device 22 (e.g., an ultrasound transducer translator) is also connected to the block 26. The device 22 is configured to be rotated upward, such that it is positioned outside of the imaging area (e.g., during x-ray imaging). In FIG. 11, the device 22 rotated into the upward position is shown in phantom. The device 22 is also configured to be rotated downward, such that it is positioned substantially parallel to, or at some angle relative to, the paddle 10 for acquisition of, for example, ultrasound images.

Generally, when using the compression paddle 10 disclosed herein, the paddle is positioned adjacent to an area of the object 16 to be subjected to a desirable medical procedure such that the plurality of filaments 14, 14′ contacts the area. The compression paddle 10 is pressed in a manner sufficient for the plurality of filaments 14, 14′ (and the frame 12) to compress the area. While the compression paddle is being pressed and the area compressed, the medical procedure is performed.

While several embodiments have been described in detail, it will be apparent to those skilled in the art that the disclosed embodiments may be modified. Therefore, the foregoing description is to be considered exemplary rather than limiting.

What is claimed is:

1. A compression paddle, comprising:
a frame defining a hollow cavity, the cavity having two opposed ends; and
a plurality of filaments attached to at least one of the two opposed ends of the cavity such that the plurality of filaments extends across the cavity in a predetermined manner.

2. The compression paddle as defined in claim 1 wherein the plurality of filaments are incorporated into a one-piece mesh material.

3. The compression paddle as defined in claim 1 wherein the predetermined manner includes at least some of the plurality of filaments crossing at least some other of the plurality of filaments to form a crisscross pattern across the cavity.

4. The compression paddle as defined in claim 1 wherein the predetermined manner includes each of the plurality of filaments aligned parallel with each of an other of the plurality of filaments.

5. The compression paddle as defined in claim 4 wherein the frame includes two opposed sides that are configured to align substantially parallel with an object, and wherein the plurality of filaments are attached to the two opposed sides such that each of the plurality of filaments are substantially perpendicular to the object.

6. The compression paddle as defined in claim 4 wherein the frame includes two opposed sides that are configured to align substantially perpendicular with an object, and wherein the plurality of filaments are attached to the two opposed sides such that each of the plurality of filaments are substantially parallel to the object.

7. The compression paddle as defined in claim 4 wherein the cavity has a substantially rectangular cross-section and is defined by two sets of opposed walls, and wherein the predetermined manner includes each of the plurality of filaments being angularly offset with respect to each of the walls of the substantially rectangular cavity.

8. The compression paddle as defined in claim 1 wherein the predetermined manner includes at least one of the plurality of filaments extending in a different direction than at least one other of the plurality of filaments.

9. The compression paddle as defined in claim 1 wherein the cavity has a substantially rectangular cross-section and is defined by two sets of opposed walls, and wherein the compression paddle further comprises a second plurality of filaments attached to at least two walls of the cavity and extending across the cavity in a second predetermined manner and at a predetermined distance from the at least one of the two opposed ends of the cavity.

10. The compression paddle as defined in claim 9 wherein the plurality of filaments extends across the cavity in a first direction and wherein the second plurality of filaments extends across the cavity in a second direction different from the first direction.

11. The compression paddle as defined in claim 1 wherein the plurality of filaments exhibit at least one of: acoustic characteristics suitable for ultrasound imaging through the paddle; or characteristics that result in minimal image effects and artifacts in medical images obtained through the paddle; or a characteristic selected from minimal absorption of ultrasound waves, an acoustic impedance that minimizes ultrasound reflection, a speed of sound that minimizes refraction, a coefficient of friction that minimizes slippage, a predetermined tensile strength, and combinations thereof.

12. The compression paddle as defined in claim 1 wherein each of the plurality of filaments has a size and shape that minimizes the presence of air bubbles and gaps between the plurality of filaments and a surface to which the plurality of filaments is exposed.

13. The compression paddle as defined in claim 1 wherein i) at least one of the plurality of filaments is spaced at least about 1 mm from an adjacent one of the plurality of filaments; or ii) at least one of the plurality of filaments is spaced a first distance from an adjacent one of the plurality of filaments, wherein at least one other of the plurality of filaments is spaced a second distance from an adjacent one of the plurality of filaments, and wherein the first distance is different than the second distance.

14. The compression paddle as defined in claim 1 wherein each of the plurality of filaments is attached such that deflection of a respective filament is minimized upon exposure to a surface.

15. The compression paddle as defined in claim 1 wherein each of the plurality of filaments is hypoallergenic, water resistant, coupling agent resistant, sterile, formed of a substantially flat material, or combinations thereof.

16. The compression paddle as defined in claim 1 wherein the compression paddle is configured for use in medical procedures selected from wire localizations, breast biopsies, hyperthermia treatments, and cryogenic treatments, and wherein the compression paddle is configured such that the medical procedure can be accomplished through the cavity of the compression paddle.

17. The compression paddle as defined in claim 1 wherein the frame is configured to tilt or flex in one or more directions.

18. The compression paddle as defined in claim 1, further comprising a grid plate or an add-on grid associated with the frame such that a predetermined area adjacent to the cavity and outlined by at least some of the plurality of filaments is identifiable via the grid plate or the add-on grid.

19. The compression paddle as defined in claim 1 wherein the plurality of filaments is integrally formed with the frame.

20. A method for using the compression paddle as defined in claim 1, the method comprising:
establishing the frame adjacent to an area to be subjected to a medical procedure such that the plurality of filaments contacts the area;
pressing the compression paddle in a manner sufficient for the plurality of filaments to compress the area; and
performing the medical procedure while the compression paddle is pressed against the area.

21. The method as defined in claim 20, further comprising:
applying a gel or liquid to the plurality of filaments and the area; and
performing the medical procedure through the cavity and between the plurality of filaments.

22. A method for making the compression paddle as defined in claim 1, the method comprising:
operatively connecting the plurality of filaments to the at least one of the two opposed ends of the cavity of the frame in the predetermined manner.

23. The method as defined in claim 22 wherein the plurality of filaments is incorporated into a one-piece mesh material, and wherein operatively connecting is accomplished by:
establishing the one-piece mesh material into slots or grooves defined between cylindrical pieces of the frame;
affixing the one-piece mesh material within the slots or grooves using a spline; and
rotating the cylindrical pieces of the frame; and

24. The method as defined in claim 22 wherein the plurality of filaments is incorporated into a one-piece mesh, and wherein the one-piece mesh is attached to the frame by:
inserting edges of the one-piece mesh into one or more grooves defined in the frame; and
affixing the mesh within the grooves using a spline.

25. The method as defined in claim 22 wherein the plurality of filaments is incorporated into a one-piece mesh material, wherein the frame includes two interlocking pieces, and wherein operatively connecting is accomplished by:
establishing the one-piece mesh material between the two interlocking pieces of the frame; and
tightening the two interlocking pieces of the frame.
